(19) United States
(12) Patent Application Publication
(10) Pub. No.: US 2026/0317411 A1
(43) Pub. Date: Oct. 15, 2026

(54) STABLE AND POTENT ANTI-ANGIOGENIC SCFV FRAGMENTS AND USES AS VEGF ANTAGONIST THEREOF

(71) Applicant: VSY BIYOTEKNOLOJI VE ILAC SANAYI ANONIM SIRKETI, Tuzla/Istanbul (TR)

(72) Inventors: Efe CAN, Tuzla/Istanbul (TR); Sibel KALYONCU UZUNLAR, Izmir (TR); Seyda GÜLLÜ, Izmir (TR); Ceren ÖZER ÇOKGEZME, Izmir (TR); Gülçin ÇAKAN AKDOGAN, Izmir (TR); Mehmet INAN, Izmir (TR); Ebru ÖNAL, Izmir (TR)

(21) Appl. No.: 19/112,058

(22) PCT Filed: Sep. 15, 2022
(86) PCT No.: PCT/TR2022/050990

§ 371 Date: Mar. 14, 2025

(51) Int. Cl. C07K 16/22

(52) U.S. Cl. CPC C07K 16/22; C07K 2317/24; C07K 2317/622

(57) ABSTRACT

A functional antibody fragment is disclosed useful as an anti-angiogenic scFv fragment and an inhibitor of VEGF:VEGFR interaction by binding to VEGF protein as a VEGF antagonist. A method is described for using the functional fragments for treating intraocular neovascular diseases. A method for the production of the antibody fragment is also provided.

### FIELD OF THE INVENTION

The present invention discloses and claims a functional antibody fragment as an anti-angiogenic scFv fragment and also as an inhibitor of VEGF:VEGFR interaction by binding to VEGF protein as a VEGF antagonist. Moreover, the present invention relates to a method of using said functional fragment for treating intraocular neovascular diseases. Pharmaceutical compositions comprising said functional fragment and methods for the preparation of a formulation are also disclosed and claimed. Furthermore, a method for the production of the antibody fragment of the present invention is provided.

### BACKGROUND

Vascular Endothelial Growth Factor (VEGF or VEGF-A) is a signal protein having important roles in angiogenesis. VEGF has been studied extensively as a critical therapeutic target for inhibiting angiogenesis. The major signaling event in angiogenesis is the binding of VEGF to VEGF receptors (VEGFRs), and the activity of VEGF serves as a rate-limiting step in the formation of normal and pathological blood vessels.

Angiogenesis is implicated in the pathogenesis of intraocular neovascular diseases, e.g., age-related macular degeneration (AMD). Age-related macular degeneration (AMD) is the most common cause of vision loss in old population, especially in the population over 50 years of age. Vascular Endothelial Growth Factor (VEGF) plays important roles in the pathogenesis of AMD. VEGF drives the development of choroidal neovascularization where new vessels grow through retinal pigment epithelium. Wet form of AMD is caused by new blood vessels leaking fluid into the retina and causing it to become wet. VEGF accumulation in wet form of AMD can be treated by anti-VEGF therapeutics. This process can be attenuated or ceased by administration of drugs into the eye (eye injection) that bind and inhibit VEGF. Currently, there are several anti-VEGF therapeutics to treat wet AMD including brolucizumab (Beovu®), aflibercept (Eylea®), ranibizumab (Lucentis®), pegaptanib sodium (Macugen®).

Single chain variable fragment (scFv) is a type of recombinant antibody. There are two variable domains (heavy and light) in an antibody structure which performs antigen binding activity. scFv consists of those two variable heavy and light chains linked by a peptide linker. They are ~25 kDa single polypeptides that contain the variable light chain (VL) and variable heavy chain (VH) of an antibody. These two chains are connected by a flexible linker peptide that is usually 15-20 amino acids long and made up of glycine (Gly or G) and serine (Ser or S) with hydrophilic residues for increased solubility [1]. It might be assumed that the variable domains of an scFv fragment would mirror that of an antibody (VL-linker-VH), but both VL-linker-VH and VH-linker-VL configurations can generate functional scFvs. Moreover, some individual scFvs performing better in one configuration than the other [2].

Because scFvs are smaller than conventional antibodies, there are many advantages for their therapeutic use such as improved tissue penetration, which is useful for therapeutic and imaging applications, rapid blood clearance which is useful for imaging applications, reduced immunogenicity when administered in vivo due to their lack of an Fc region, and less challenging production process in microbial systems due to lack of glycosylation needs. Furthermore, scFvs are cheaper and easier to make because they can be expressed in microbial systems, while mAbs generally require mammalian expression systems [3, 4].

Compared to antibodies, scFvs might have some drawbacks related to their stability, production yields, potency, and activity. scFvs tend to have lower long-term stability, lower affinities, and a higher likelihood to aggregate due to their small size [4]. The generation of functional scFvs is still limited by these disadvantages.

The United States patent document no. U.S. Pat. No. 9,149,427B2 discloses a cell line comprising an ARPE-19 cell genetically engineered to produce a therapeutically effective amount of one or more anti-angiogenic polypeptides or anti-angiogenic molecules. In this document, novel cell lines are described without any claims of technical superiority of scFv proteins. The United States patent document no. U.S. Pat. No. 8,936,785B2 discloses a new therapeutic route of eye drops for the said scFv proteins. scFvs might have some drawbacks related to their stability, production yields and activity. There is no explanation for in vitro and in vivo advantages of these scFv proteins regarding these technical problems.

Accordingly, there has been still a need to design a new anti-VEGF antagonist based on scFv fragments that overcome the technical issues mentioned above. These needs and other needs are satisfied by the present invention. The present invention relates to scFv sequences showed technical advantages: (i) higher thermal stability, (ii) higher production yield and (iii) more potent anti-angiogenic activity in in vivo zebrafish animal models.

### SUMMARY OF THE INVENTION

Accordingly, a broad embodiment of the invention is directed to a stable, soluble and potent VEGF-binding functional antibody fragment.

According to a first aspect of the invention there is provided a functional fragment thereof that specifically binds to a Vascular Endothelial Growth Factor (VEGF) protein.

Another aspect of the present invention relates to a functional fragment wherein said functional fragment has (i) high thermal stability, (ii) high production yield and (iii) highly potent anti-angiogenic activity in in vivo zebrafish models.

The other aspect of the present invention is to provide to a functional fragment for treating and/or preventing an intraocular neovascular diseases or disorders, wherein the fragment is characterized by binding to and inhibiting vascular endothelial growth factor A (VEGF).

The invention can be used for the preparation of a medicament (pharmaceutical composition) useful in the treatment and/or prevention of disorders due to the inhibition of VEGF:VEGFR interaction and the anti-VEGF activity.

Yet another objective of the present invention is to provide a pharmaceutical composition comprising a pharmaceutical carrier and a therapeutically effective amount of the functional antibody fragment.

In a further aspect, the present invention relates to a method provided of applying a pharmaceutical composition to a subject and a pharmaceutical composition of administering it to a subject is provided.

Another embodiment of the present invention relates to a method and a pharmaceutical composition for the treatment of age-related macular degeneration (AMD).

Accordingly, a broad embodiment of the invention is directed to an anti-VEGF therapy method which is the use of medications that block vascular endothelial growth factor. This is done in the treatment of certain cancers and in age related macular degeneration. The anti-VEGF therapy method can be used for clinical applications for ocular disorders and systemic diseases where VEGF plays a primary role.

Furthermore, a method for the production by microbial fermentation of the antibody fragment of the present invention is provided.

This object and other objects of this invention become apparent from the detailed discussion of the invention that follows.

### BRIEF DESCRIPTION OF FIGURES

The present invention is illustrated in the accompanying figures wherein;

FIG. 1 is a schematic showing the vector map (genes are purchased from GenScript and cloned into pPICZαA vector).

FIG. 2 shows the production of scFv in fermentor at different time points.

FIG. 3 is an illustration of thermal stability analysis of scFv proteins.

FIG. 4 is an illustration of SE-HPLC and SDS-PAGE profiles of scFv proteins (scFv1, scFv2, scFv3) with comparison to reference scFv. Their purity percentages are also indicated.

FIG. 5 is SPR sensograms for scFv proteins (scFv1, scFv2, scFv3) with comparison to reference scFv

FIG. 6 is an illustration of in vivo potency analysis of scFv proteins (scFv1, scFv2, scFv3) with comparison to reference scFv in zebrafish angiogenesis model.

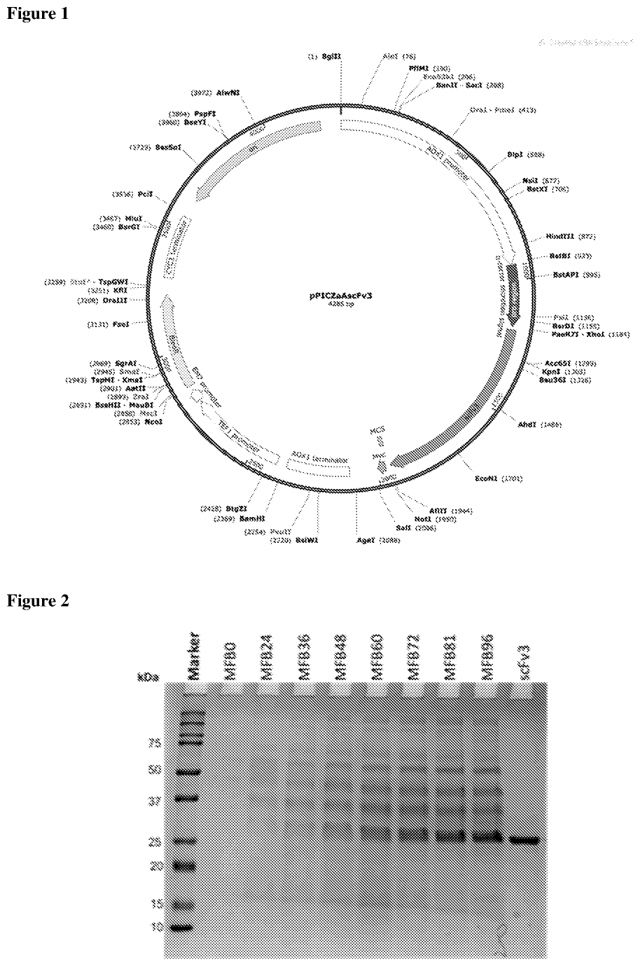

### DETAILED DESCRIPTION OF THE INVENTION

The present invention relates to the fields of functional antibody fragments, their production by microbial fermentation, their solubility/stability/binding characterizations, and their in vivo tests in zebrafish models.

Accordingly described herein, the term “functional fragment” or “functional antibody fragment” refers to fragments of Fv, scFv, Fab, F(ab′)2, F(ab′), scFv-Fc type or diabodies. In a preferred embodiment, the functional antibody fragment is a single chain variable fragment (scFv).

Furthermore, the invention provides a process for the production of the antibody construct of the invention, a medical use of said antibody construct and a kit comprising said antibody construct as defined. The antibody construct which comprises scFv as a functional fragment is in a format selected from the group consisting of (scFv)2, scFv-single domain mAb, diabodies and oligomers of the foregoing formats.

As used herein, the term “inhibit”, “inhibition” or “inhibiting” refers to the reduction or suppression of a given condition, symptom, or disorder, or disease, or a significant decrease in the baseline activity of a biological activity or process such as VEGF:VEGFR interaction. Inhibitors of VEGF:VEGFR interaction are promising candidates for the treatment of systemic diseases, cancers (solid tumors) and various ocular disorders.

The present invention discloses and claims a functional antibody fragment as an anti-angiogenic scFv fragment and also as an inhibitor of VEGF:VEGFR interaction by binding to VEGF protein as a VEGF antagonist. Furthermore, it is a humanized monoclonal single-chain Fv (scFv) antibody fragment targeting VEGF protein.

According to the present invention, the single-chain variable fragment (scFv) comprises a variable heavy chain (VH) and a variable light chain (VL) linked by a peptide linker.

Also, peptide linker choice and sequence lineup of variable heavy/light chains are important factor for in vitro and in vivo properties of scFvs. In the present invention, an optimal domain orientation and a flexible peptide linker is developed with optimal content and length.

According to the present invention, the single-chain variable fragment (scFv) comprises scFv1, scFv2 and scFv3, respectively:
a first binding domain which contains in sequence a variable light chain with the amino acid sequence of SEQ ID NO:1, a linker with the amino acid sequence of SEQ ID NO:2, a second binding domain which contains in sequence a variable heavy chain with the amino acid sequence of SEQ ID NO:3; or
a first binding domain which contains in sequence a variable light chain with the amino acid sequence of SEQ ID NO:4, a linker with the amino acid sequence of SEQ ID NO:5, a second binding domain which contains in sequence a variable heavy chain with the amino acid sequence of SEQ ID NO:6; or
a first binding domain which contains in sequence a variable light chain with the amino acid sequence of SEQ ID NO:7, a linker with the amino acid sequence of SEQ ID NO:8, a second binding domain which contains in sequence a variable heavy chain with the amino acid sequence of SEQ ID NO:9.

In the first embodiment, the single-chain variable fragment (scFv) comprises scFv1 with the amino acid sequence of SEQ ID NO:10 which consists of SEQ NO:1, SEQ NO:2 and SEQ NO:3, respectively.

In the second embodiment, the single-chain variable fragment (scFv) comprises scFv2 with the amino acid sequence of SEQ ID NO:11 which consists of SEQ NO:4, SEQ NO:5 and SEQ NO:6, respectively.

In the third embodiment, the single-chain variable fragment (scFv) comprises scFv3 with the amino acid sequence of SEQ ID NO:12 which consists of SEQ NO:7, SEQ NO:8 and SEQ NO:9, respectively.

Primary structure of these scFvs (scFv1, scFv2, scFv3) are the same but only the four amino acids marked for each sequence (as shown in the sequence list) can be changed in different combinations (F (Phenylalanine; Phe) to amino acid E (Glutamic acid; Glu) or vice versa at position 83, S (Serine; Ser) to amino acid M (Methionine; M) or or vice versa at position 12).

Accordingly, the present invention relates to a scFv (scFv1, scFv2, scFv3) that specifically binds to a Vascular Endothelial Growth Factor (VEGF) protein, wherein said scFv has a high binding affinity to VEGF protein, and wherein the scFv thereof binds the VEGF protein with a dissociation constant (K_{D}) of less than 5 nM as measured by surface plasmon resonance (SPR). In a preferred embodiment, the dissociation constant is less 1 nM, more precisely between 0.01 nM and 1 nM.

In further embodiment, the invention provides scFv that binds VEGF protein with an association rate constant (kₒₙ) of 7×10⁴ M⁻¹s⁻¹ or greater.

Moreover, the invention provides scFv that binds VEGF protein with a dissociation rate constant (k_{off}) of 3×10⁻⁶ s⁻¹ or lower.

Dissociation constant (K_{D}), association rate constant (kₒₙ) and dissociation rate constant (k_{off}) values were calculated by fitting the kinetic association and dissociation curves to a 1:1 binding model.

In another embodiment, the invention provides an anti-VEGF scFv thereof according to any one of the preceding embodiments, wherein the VEGF protein is a mammalian VEGF protein; more specifically it is a human VEGF protein (Gibco Catalog #PHC9391).

Furthermore, a method for the production of the antibody fragment of the present invention is provided.

According to the invention, a method for the production of scFv antibody fragment is provided by microbial fermentation process which comprises the step of:
culturing of the host cell transformed with the DNA encoding said scFv under conditions that allow the expression and production of said fragments,
inoculating the culture to a bioreactor to produce recombinant scFvs at a large-scale level under suitable conditions by a step-by-step scale up method,
adjusting the pH with a pH adjusting agent which also served as a nitrogen source,
depletion of glycerol in the culture (dissolved oxygen spike),
after the dissolved oxygen spike signal, starting the fed-batch phase of fermentation feeded by 100% methanol or ethanol or glucose containing trace metal solution,
increasing the feed rate of methanol or ethanol or glucose solution exponentially during the first 72 hours of the fermentation and then holding the rate constant during the last 24 hours of the fermentation.
optimization of the temperature and pH during the batch phase,
and recovering said supernatants from said fermented culture at different times.

The present invention relates to a pharmaceutical composition comprising a humanized anti-VEGF scFv as described in any one of the preceding embodiments for use in anti-VEGF therapy by inhibiting VEGF:VEGFR interaction in a mammal, wherein said medicament can be administered in a therapeutically effective amount to the mammal. Preferably, the mammal is human, more preferably the mammal is a human with a tumour (solid tumour; cancer) or with an ocular disorder.

The present invention relates to a pharmaceutical composition comprising a therapeutically effective amount of scFv as described in any one of the preceding embodiments and a pharmaceutically acceptable carrier. The composition is an aqueous solution for ophthalmic applications. More preferably, the pharmaceutical composition is an ophthalmic solution containing phosphate buffered saline administered as an eye drop or eye injection.

It is found that anti-VEGF effect of the scFv antibody fragment is concentration-dependent. The concentration of scFv is in the range of 20 μM to 100 μM.

The term “a therapeutically effective amount” of a functional fragment of the present invention refers to a non-toxic and sufficient amount of the fragment of the present invention that will elicit the biological or medical response of a subject, for example, reduction or inhibition of the protein:protein or protein:receptor interaction, or ameliorate symptoms, alleviate conditions, slow or delay disease progression, or prevent a disease or disorder, etc.

The pharmaceutical composition of the invention as described in any one of the preceding embodiments can be used for the treatment of any VEGF protein associated diseases or disorders such as intraocular neovascular diseases.

In another embodiment, the pharmaceutical composition of the invention as described in any one of the preceding embodiments can be used for the treatment of age-related macular degeneration (AMD), more preferably for the treatment of wet form of age-related macular degeneration (wet AMD).

In a further embodiment, the present invention relates to a method provided of applying the pharmaceutical composition to a subject and a pharmaceutical composition of administering it to a subject is provided.

All of the various embodiments of the present invention as disclosed herein relate to methods of treating and/or preventing intraocular neovascular diseases or disorders as described herein.

Non-limiting examples of ocular neovascular diseases include age-related macular degeneration (AMD), wet form of age-related macular degeneration (wet AMD), diabetic retinopathy, retinal vascular occlusion, retinopathy of prematurity.

These examples are intended as representative of specific embodiments of the invention and are not intended as limiting the scope of the invention.

### Specific Embodiments

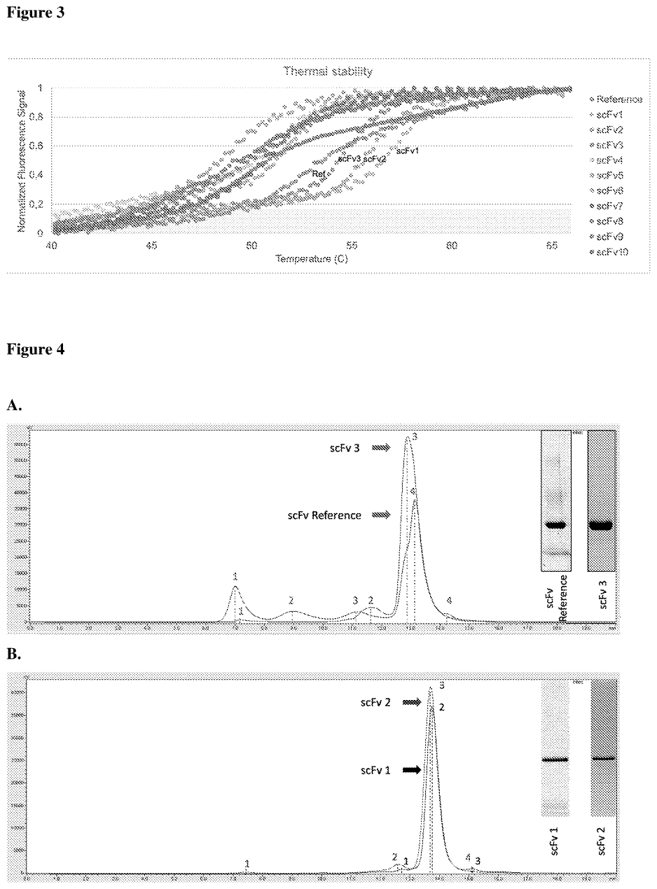

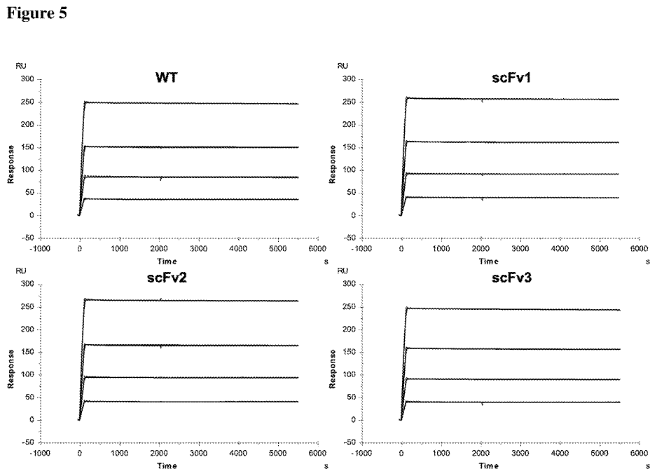

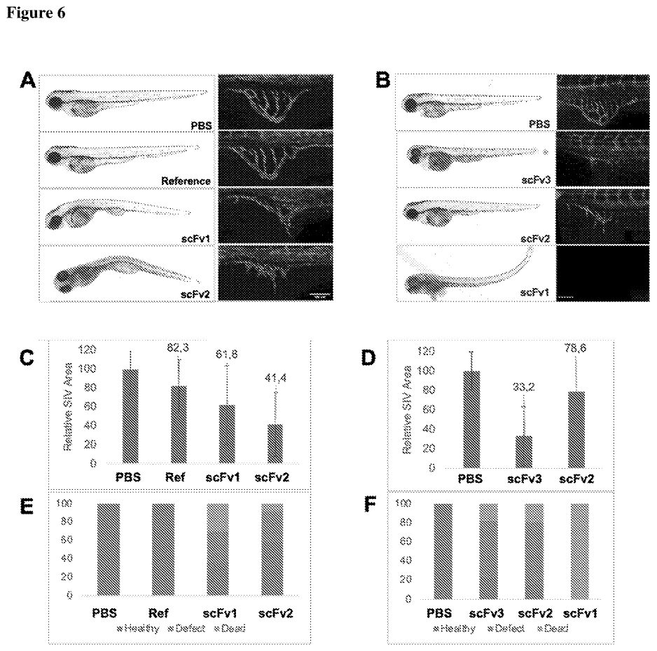

Stability, production yield and potency are very important for scFv development. In these embodiments, we engineered some scFv proteins to obtain highly stable, high yield and highly potent scFv proteins for therapeutic applications. Compared to our reference antibody, three scFv proteins (scFv1, scFv2, scFv3) gave advantageous properties. Protein yield of scFv3 was nearly 10 times more than reference with >200 mg purified scFv (MW: ~26.8 kDa) per liters of culture (FIG. 2). scFv1 and scFv2 gave nearly 5° C. higher thermal stability than reference (FIG. 3). After only one step Protein L affinity chromatography, scFv1 and scFv2 gave 97.3%, 93.3% purity, respectively while reference gave 65% purity (Table 1, FIG. 4). According to surface plasmon resonance results, scFv1, scFv2 and scFv3 bind to VEGF stronger than reference (Table 2, FIG. 5). A transgenic zebrafish model where GFP expression was induced in endothelial cells was used for anti-angiogenesis potency test. It was shown that scFv1, scFv2 and scFv3 gave higher efficacy than reference while scFv3 gave the highest in vivo activity among them by 76.8% decrease in SIV area (FIG. 6).

TABLE 1
Purity analysis of scFv proteins by SE-HPLC

| | Peak | RT | Area | Peak % |
|---|---|---|---|---|
| scFv | 1 | 7.00 | 422654 | |
| Reference | 2 | 8.98 | 259000 | |
| | 3 | 11.17 | 235113 | |
| | 4 | 13.14 | 1703230 | 65% |
| | (Main peak) | | | |
| scFv3 | 1 | 7.17 | 14852 | |
| | 2 | 11.66 | 239271 | |
| | 3 | 12.88 | 2445682 | 88.6% |
| | (Main peak) | | | |
| | 4 | 14.05 | 61602 | |
| scFv1 | 1 | 12.75 | 12820 | |
| | 2 | 13.74 | 1133723 | 97.3% |
| | (Main peak) | | | |
| | 3 | 15.12 | 18578 | |
| scFv2 | 1 | 7.44 | 6614 | |
| | 2 | 12.60 | 82313 | |
| | 3 | 13.70 | 1370629 | 93.3% |
| | (Main peak) | | | |
| | 4 | 14.93 | 8332 | |

To sum up, we engineered anti-VEGF scFv fragments with enchanced:
1. Thermal stability (>5° C. increase in thermal stability)
2. Higher production yields in microbial systems (>200 mg purified scFv per liters of culture). Also, we got more than 95% purity after one step purification.
3. Higher anti-angiogenic efficacy in in vivo zebrafish models (more than two-fold efficacy)

### EXAMPLES

### Example 1—Production

Komagataella phaffii (Pichia pastoris) is used to express and produce soluble scFv proteins. The 5-L bioreactor was used to produce recombinant scFvs at a large-scale level. A vial of frozen culture was inoculated into 200 mL media and incubated at 28° C., 225 rpm for approximately 20 hours. 125 mL of the culture was inoculated into 2.5 L media. pH was adjusted with 25% ammonium hydroxide which also served as a nitrogen source. The batch phase was continued for about 15 hours and completed with depletion of glycerol in this media (dissolved oxygen spike). After the dissolved oxygen spike signal, the fed-batch phase of fermentation was started feeding by 100% methanol or ethanol or glucose containing trace metal solution. The feed rate was exponentially increased during the first 72 hours of fermentation and this rate was held constant during the last 24 hours of the fermentation. The temperature and pH during the batch phase were optimized at 18-30° C. and 3.0-7.0, respectively. The temperature and pH values were set linearly with profile in the first hour of the fed-batch phase. Dissolved oxygen level was controlled at the 30% saturation by adding air and pure oxygen with constant agitation. During the fed-batch phase, the supernatants were collected at different times. These samples were analyzed to measure the biomass level and determine produced protein with SDS-PAGE.

### Example 2—Purification

Filtered supernatant was applied onto an affinity chromatography column (Protein L). More than 93% purity was achieved by this first step purification. Second step purifications were applied to obtain >95% purity.

### Example 3—Size-Exclusion Chromatography (SE-HPLC)

An HPLC System with UV-VIS detector and an analytical size-exclusion chromatography column were used to perform SE-HPLC. scFv samples were injected into the column. The absorbance values were monitored at 280 nm. Molecular weight standards were used to verify the retention times of scFv proteins.

### Example 4—Thermal Stability Assay

Thermal unfolding profiles of purified scFv proteins were determined with a fluorescent dye. Optimum concentrations of dye and protein were used. Transition mid-points (Tₘ values) from the thermogram data were calculated based on an equation.

### Example 5—Surface Plasmon Resonance (SPR)

Affinity measurements were performed using a Biacore instrument. VEGF protein was immobilized onto a chip. A series of solutions ranging from 1 to 100 nM scFv proteins were subsequently injected onto the VEGF-coated surface. Data were corrected by double-referencing against a control flow cell containing no VEGF and against the flow cell with buffer injection. Sensogram curves were analyzed. Dissociation constant (K_{D}), association rate constant (kₒₙ) and dissociation rate constant (k_{off}) values were calculated by fitting the kinetic association and dissociation curves to a 1:1 binding model.

TABLE 2
Binding kinetics of scFv proteins by surface plasmon resonance.

| | K_{D} | kₒₙ | k_{off} |
|---|---|---|---|
| Sample | (pM) | (×10⁴ M⁻¹s⁻¹) | (×10⁻⁶s⁻¹) |
| Reference | 26.1 ± 8.2 | 7.646 ± 0.002 | 1.990 ± 0.008 |
| scFv* | | | |
| scFv1 | 20.5 ± 4.4 | 9.094 ± 0.002 | 1.870 ± 0.004 |
| scFv2 | 17.2 ± 6.0 | 8.793 ± 0.001 | 1.520 ± 0.006 |
| scFv3 | 23.6 ± 7.8 | 9.756 ± 0.006 | 2.300 ± 0.005 |

*10 sequences are shown in sequence list as SEQ ID No 10-19, respectively. The reference sequence is SEQ ID No 20.

### Example 6—Zebrafish Experiments

Zebrafishes used in this study were provided by the Zebrafish facility in Izmir Biomedicine and Genome Center. All animal procedures were approved by the ethical committee (HADYEK) of Izmir Biomedicine and Genome Center, Izmir, Turkey. In addition, all experiments involving live animals were performed in accordance with relevant guidelines and regulations and were reported as described by the recommendations in the ARRIVE guidelines.

Adult zebrafishes were maintained under standard conditions at the Izmir Biomedicine and Genome Center Zebrafish Facility. A transgenic line was crossed to wild type to obtain the embryos.

The estimated injection volume was determined by the use of a stage micrometer. scFv proteins were microinjected into the yolk of each anesthetized zebrafish embryo at 2 days post fertilization. Microinjected larvae were kept at 28° C. and at 1-day post-injection. Development of subintestinal vessels (SIV) were examined. Larvae were fixed and melanocytes were decolorized with pigment discoloration solution.

Larvae were mounted laterally in low melting agarose in imaging dish, and imaged with a confocal microscope. SIV area was measured and statistical analysis was performed.

### REFERENCES

[1]. Monnier P, Vigouroux R, Tassew N (2013) In Vivo Applications of Single Chain Fv (Variable Domain) (scFv) Fragments. Antibodies 2:193-208.
[2]. Sandomenico A, Sivaccumar J P, Ruvo M (2020) Evolution of Escherichia coli Expression System in Producing Antibody Recombinant Fragments. IJMS 21:6324.
[3]. Ahmad Z A, Yeap S K, Ali A M, Ho W Y, Alitheen N B M, Hamid M (2012) scFv Antibody: Principles and Clinical Application. Clinical and Developmental Immunology 2012:1-1.
[4]. Bates A, Power C A (2019) David vs. Goliath: The Structure, Function, and Clinical Prospects of Antibody Fragments. Antibodies 8:28.

1. A single-chain variable fragment (scFv) that specifically binds to a Vascular Endothelial Growth Factor (VEGF) protein, wherein scFv has a high binding affinity to VEGF protein, comprising:
a first binding domain which contains in sequence a variable light chain with the amino acid sequence of SEQ ID NO:1, a linker with the amino acid sequence of SEQ ID NO:2, a second binding domain which contains in sequence a variable heavy chain with the amino acid sequence of SEQ ID NOG; or
a first binding domain which contains in sequence a variable light chain with the amino acid sequence of SEQ ID NO:4, a linker with the amino acid sequence of SEQ ID NOG, a second binding domain which contains in sequence a variable heavy chain with the amino acid sequence of SEQ ID NOG; or
a first binding domain which contains in sequence a variable light chain with the amino acid sequence of SEQ ID NO:7, a linker with the amino acid sequence of SEQ ID NO:8, a second binding domain which contains in sequence a variable heavy chain with the amino acid sequence of SEQ ID NO:9.

2. The single-chain variable fragment according to claim 1, wherein the VEGF protein is mammalian VEGF protein.

3. The single-chain variable fragment according to claim 2, wherein the mammalian VEGF protein is human VEGF protein.

4. The single-chain variable fragment according to claim 3, wherein the scFv binds the VEGF protein with a dissociation constant of less than 5 nM.

5. The single-chain variable fragment according to claim 4, wherein the scFv binds the VEGF protein with a dissociation constant of less than 1 nM.

6. The single-chain variable fragment according to claim 5, wherein the scFv binds the VEGF protein with an association rate constant of 7×10⁴ M″ greater.

7. The single-chain variable fragment according to claim 6, wherein the scFv thereof binds the VEGF protein with a dissociation rate constant (kOff) of 3×10-6 s′1 or lower.

8. A pharmaceutical composition comprising a humanized anti-VEGF scFv according to claim 1 for anti-VEGF therapy by inhibiting VEGF:VEGFR interaction in a mammal, wherein said medicament can be administered in a therapeutically effective amount to the mammal.

9. The pharmaceutical composition according to claim 8, wherein the mammal is human.

10. The pharmaceutical composition according to claim 9, wherein the mammal has a tumor.

11. The pharmaceutical composition according to claim 9, wherein the mammal has an ocular disorder.

12. The pharmaceutical composition comprising a pharmaceutically acceptable carrier and a therapeutically effective amount of scFv according to claim 1.

13. The pharmaceutical composition according to claim 12, wherein the pharmaceutical composition is an ophthalmic aqueous solution.

14. The pharmaceutical composition according to claim 13, wherein the composition is for the treatment and/or prevention of age-related macular degeneration (AMD).

15. A method of treating or preventing age-related macular degeneration (AMD) comprising administering to a subject an effective amount of a scFv according to claim 1.

16. An antibody construct comprising a scFv according to claim 1, wherein the antibody construct is in a format selected from the group consisting of (scFv)2, scFv-single domain mAb, diabodies and oligomers of the foregoing formats.


SEQUENCE LISTING (20 sequences)

SEQ ID NO: 1 - protein, 107 aa, synthetic construct
DIQLTQSPSSLSASVGDRVTITCSASQDISNYLNWYQQKPGKAPKVLIYFTSSLHSGVPSRFSGSGSGTDFTLTISSLQPEDEATYYCQQYSTVPWTFGQGTKVEIK

SEQ ID NO: 2 - protein, 20 aa, synthetic construct
GGGSGGGSGGGSGGGSGGGS

SEQ ID NO: 3 - protein, 123 aa, synthetic construct [sequence omitted]

SEQ ID NO: 4 - protein, 107 aa, synthetic construct
DIQLTQSPSSLMASVGDRVTITCSASQDISNYLNWYQQKPGKAPKVLIYFTSSLHSGVPSRFSGSGSGTDFTLTISSLQPEDFATYYCQQYSTVPWTFGQGTKVEIK

SEQ ID NO: 5 - protein, 20 aa, synthetic construct
GGGSGGGSGGGSGGGSGGGS

SEQ ID NO: 6 - protein, 123 aa, synthetic construct [sequence omitted]

SEQ ID NO: 7 - protein, 107 aa, synthetic construct
DIQLTQSPSSLMASVGDRVTITCSASQDISNYLNWYQQKPGKAPKVLIYFTSSLHSGVPSRFSGSGSGTDFTLTISSLQPEDEATYYCQQYSTVPWTFGQGTKVEIK

SEQ ID NO: 8 - protein, 20 aa, synthetic construct
GGGSGGGSGGGSGGGSGGGS

SEQ ID NO: 9 - protein, 123 aa, synthetic construct [sequence omitted]

SEQ ID NO: 10 - protein, 250 aa, synthetic construct
DIQLTQSPSSLSASVGDRVTITCSASQDISNYLNWYQQKPGKAPKVLIYFTSSLHSGVPSRFSGSGSGTDFTLTISSLQPEDEATYYCQQYSTVPWTFGQGTKVEIKGGGSGGGSGGGSGGGSGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSS

SEQ ID NO: 11 - protein, 250 aa, synthetic construct
DIQLTQSPSSLMASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGGGSGGGSGGGSGGGSGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSS

SEQ ID NO: 12 - protein, 250 aa, synthetic construct
DIQLTQSPSSLMASVGDRVTITCSASQDISNYLNWYQQKPGKAPKVLIYFTSSLHSGVPSRFSGSGSGTDFTLTISSLQPEDEATYYCQQYSTVPWTFGQGTKVEIKGGGSGGGSGGGSGGGSGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSS

SEQ ID NO: 13 - protein, 250 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGGGSGGGSGGGSGGGSGGGSERQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSS

SEQ ID NO: 14 - protein, 250 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGGGSGGGSGGGSGGGSGGGSEVQLVESGGGLVQPGGSLRLSCAASGYDFTHYGMNWVRQEPGKGLEWVGWINTYTGEPTYAADFKRRFTFSLDTSKSTAYLQMNSLRAEDTAVYYCAKYPYYYGTSHWYFDVWGQGTLVTVSS

SEQ ID NO: 15 - protein, 250 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGGGSGGGSGGGSGGGSGGGSEVQLVESGGGLVQPGGSLRLSCAASGYDFTHYGMNWVRQAPGKGLEWVGWINTYTGEPTYAADFKRRFTFSLDTSKSTAYLQMRSLRAEDTAVYYCAKYPYYYGTSHWYFDVWGQGTLVTVSS

SEQ ID NO: 16 - protein, 250 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGGGSGGGSGGGSGGGSGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDYWGQGTLVTVSS

SEQ ID NO: 17 - protein, 250 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGGGSGGGSGGGSGGGSGGGSEVQLVELGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSS

SEQ ID NO: 18 - protein, 250 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGGGSGGGSGGGSGGGSGGGSEVQLVESGGGLVQPGGSLRLSCAASGYDFTHYGMNWVRQAPGKGLEWVGWINTYTGEPTYAADFKRRFTFSLDTSKSTAYLSMNSLRAEDTAVYYCAKYPYYYGTSHWYFDVWGQGTLVTVSS

SEQ ID NO: 19 - protein, 250 aa, synthetic construct
DIQLTQSPSSLSASVGDRVTITCSASQDISNYLNWYQQKPGKAPKVLIYFTSSLHSGVPSRFSGSGSGTDFTLTISSLQPEDEATYYCQQYSTVPWTFGQGTKVEIKGGGSGGGSGGGSGGGSGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDYWGQGTLVTVSS

SEQ ID NO: 20 - protein, 250 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGGGSGGGSGGGSGGGSGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSS